SEQUENCE LISTING (31 sequences)

SEQ ID NO: 1 - DNA, 1455 bp, Rhizopus delemar
  CDS (1)..(1455)
atgaccaagaacgttcttactacatccaatggtaatcctgtcgaaaacaaccaaacatctcaaactgctggtgcctggggacctgtccttattcaagattttcatttagttgataaacttgctcactttgacagagaacgtattcctgagcgtgtggttcatgcaaaaggtgctggtgcacacggtgtttttgaggttactaatgacatcactcatctcaccaaggccaagtttttgagtcatgttggtaaaacaactccagtctttcttcgattctcaactgttggtggtgaaaaaggtagtgctgatactgctcgtgatcctcgcggttttgctttaaaattttatactgaagaaggcaattgggatatggttggcaataataccccagtcttctttattcgtgatccttccaagttcccagactttattcacactcaaaaaagaaaccctagaaccaactatgctgatccagatgccttttgggatttcctttcccttgttcccgagtcaattcatcaagtcaccattcttttctccaacagaggtacacctgatggttaccgccatctcaatggctactcctctcacactctcaagttggtcaacgataagggcgagttcaagtacgttaaatggcacttcaagaccgatcaaggcatcaagaatctcacagcccaaaaggctggtgaacttgctggctctgatccagactacgccacacgcgaccttttcaatgccattgaacgcggtgattatccttcttggtctgtttatatccaagtcatgaaccctgaagatgccaaaaagtatcgcttcaatcctttcgacgttaccaaagtttggtcacacaaggattatcctcttcaacctgtgggcaaacttacattgaaccgtaaccctgaaaactactttgctgagactgagcaagctgccttctctccttctcatattgtacctggtattgatgtttctcctgatcgtatgttgcaaggccgtctcttttcttaccctgatactcaccgtcatcgtcttggtgtgaactatgcacagattcctattaaccaaccccagtgccgtgtctctaatcatcagcgtgatggtcaaatggctgttttgggcaatggtggatctgctcctaattatgaacccaactcattcagtggacctcaacaaaccaatattgccgctactacttttaccgctgaagaacttgaaggcgtcactggccgtcatagttttaccttgaccgacgatgactttgtacaagctggtgacttgtatcgtctcatgtctgctgaagaaaagactgatttgatcaacaatatcgccaggcacttgaagaatgccaagaagcatatccaagagcgccaaattggtcactttaagcgtgctgatccagaatatggtcaaagaattgagcagactattttggctttgagctcaaaagcttaa

SEQ ID NO: 2 - protein, 484 aa, Rhizopus delemar
MTKNVLTTSNGNPVENNQTSQTAGAWGPVLIQDFHLVDKLAHFDRERIPERVVHAKGAGAHGVFEVTNDITHLTKAKFLSHVGKTTPVFLRFSTVGGEKGSADTARDPRGFALKFYTEEGNWDMVGNNTPVFFIRDPSKFPDFIHTQKRNPRTNYADPDAFWDFLSLVPESIHQVTILFSNRGTPDGYRHLNGYSSHTLKLVNDKGEFKYVKWHFKTDQGIKNLTAQKAGELAGSDPDYATRDLFNAIERGDYPSWSVYIQVMNPEDAKKYRFNPFDVTKVWSHKDYPLQPVGKLTLNRNPENYFAETEQAAFSPSHIVPGIDVSPDRMLQGRLFSYPDTHRHRLGVNYAQIPINQPQCRVSNHQRDGQMAVLGNGGSAPNYEPNSFSGPQQTNIAATTFTAEELEGVTGRHSFTLTDDDFVQAGDLYRLMSAEEKTDLINNIARHLKNAKKHIQERQIGHFKRADPEYGQRIEQTILALSSKA

SEQ ID NO: 3 - DNA, 2298 bp, Rhizopus delemar
atgaccactttacttattgacaactacgacagttttacttataatgtctatcaatacttgagctgccaaggcgccaatgtagttgtctacagaaacgacaaaatcaccatttccgaaattgagcaattggctcctcgcaatattgtcatctcacctggccctggccacccttccaccgatgccggtgtctctcgagaggccattcgagcttttgcaggaaagattcccatcttgggtatttgtatgggtcagcaatgtatgtatgaagtgtacggtggtaaagtgtcatatgcaggtgatattgtgcatggcaaggcatccagcatcaagcatgacagtcgaggtatcttcaagggcgttcctcaaaacaacatggtcactcgttaccattcccttgctggcatgccttctactttacctgaaacattagaagtcactgcgactaccgacgatggtatcatcatgggcattcgacacaaggaatacactgtcgaaggtgttcagttccatcctgaaagtatcctttgtgaacacggacatacgatgatcaacaacttcttaagcttgcgtggtggcacctgggaagagaatcctgcagccggtgttgtctttaagaaagctcgttccgaaacacccaaaatcagtgctagtgaatcccaactcgatctctctcagcaacaacctgccgcagcaccttccatcttgacccgcatttactctcaacgactcaaggatgttcaggcagccaaggagattcccggccagacatttgaagatttagaaaaacttttaaagttgcacgtcgccccacctcttcaagacgtcgtcgctcgcgtgcgtcaaagcaagcccgccttgatggccgaagtcaagcgtgcctctccctcgaaaggaaacattgatgtttcggccaacgcggctgagcaggcacttcaatatgctttagcaggtgcaagcgtcgtctctgttctgactgaacccaaatggttccgcggtacgattcatgatatgcatcaggtccgagaggccttgagccatctgcccaaccgtccttgtgtgttgagaaaggattttattgtcgatcgctatcaaatcttggaaggttgtctgtacggtgctgatactatcttgttgatcgtggccatgctgaatgatgaacaactgcacgaattgtatcactatgcgaaatcattaggtatggaacccttggtcgaagtcaataatacggaagagatggcccgtgccaatgctttgggcgcacgtctggtgggtgttaataatcgcaacttgcacagctttgatgttgatatggaaaccacgagtcgattggtagagatggtgcctgaaggaacgatcttgtgtgcactttctggtattactggacgagctgatgttgaaatgtacgtcaaacagggtgtgcacgctgtcttggtgggtgaagccctgatgcgtgcttggaatttgaaggagtttgtgtctgatttgttgggtcatgaaaagaaggatcctgtgcctgtgtccaaggaatcaaaatcttcactagtcaaggtatgtggtatctctagtgtggatgcagcagttgaagcagccaagtcaggggctgacttgattggtcttatctttgctgaaaagtccaaacgaaaagtgtctttggaaagagctcaagaaatcgtgtcctcagtgcgtgcgttggatattcaagtcaaacgaacgttatcaaatgatgattctcaactggattggttccagatgcacaagcgtctcttggaaaagcgagcaagaaaacctttggtagttggcgtgtttgtgaatcaatcgattgaatacatgactgaggtggcaacgacagtcggactggaccttattcagctgcatggaaccgaatcaacggagcttgcacgctatttacccgtgcctgtcatcaaagctttccatatcgacagtggtgagttcaatgaagctcagataccaaacctaaatcaaccaggctcttatcattatgtcttactggacgctaaagtgcccagcttaccatcggatcaacaaggtggacgtggtgtcaagtttgattggtcaattgctaccaaaatcgtgaaacataggcactttgagtttttgggtaatcaagatttccctgtcatcttggctggtgggttggatcctaccaatgtggcatctgccattcaacaggtgaaaccctggattgtggatgtgtcgagtggtgttgaaacagatggagtgaaggatttagaaaagattcgtgcctttgttaaaactgtccagtcaacacaattttaa

SEQ ID NO: 4 - DNA, 1000 bp, Rhizopus delemar
tagagggaaaaagagagaattgaaataggagaggatgagtcaaaatatagtttacataaaatttctcttttttgtgttaaatataatctaatagcaggggttttcttagtttacgtttatatcaaagttatcaagcatacacttttttatgatttttcatactttaatcccttttagtattctattgtttgaaaggagagaaaaaacagctgagggtacggtgcacacgagatcttacgataattttcctgcccaacaggaaagaagtaattgatcttgattgacgctcggagtttgcacgttcggagtttgcacttcacattgagttatactcttacttattttgaaggaagggacgagaaaagatgtaaatataataataacagtagtaaatagtatgcgcatcaagaacagctaccaacaaaagagagaaatatgagcttaataatgaacaatgtaaatggcagaatgaaatttaattatcaaagcggcatctttcagaccttccgttacttccgatagagttttttatgcaaagtaataacaactgtatatataaaaaaaagaaggttatcaagcaaaagccacaatgtcatatctggaataatcaagagtaactattgaatgttggtagccaaaagaggcacgtaattttatgacgaaatatcacacaaaaagattattttgacaattcatgaataggacagagatacaccctaaacatgaaatgtaagctatatttaaacacctcaagttaattttgaagcttcatttgtattattgtaaccatttagacaagctaaatcctttttattattgtccttattgattttatccagattaccgtatctaaagagcgatcaacagaaaaacggctgattttagaccaaagtttcacaaactacatttgcatgaacgtcatatatatataaaccttgacttttcttttttttttttttttttttttttcattatcaattaatacaattaaataacaaaa

SEQ ID NO: 5 - DNA, 634 bp, Rhizopus delemar
tcattttaattacgcattttcatttactaatttgttacattttgataacgtcaataaatcttctaatttcttttgttctcaaacagtttacagttctatctttttttttaccacaatcaattctcaatatacaacatagcaaatgtgcttcagtaaattcattaaattcttttaaaaaaaggtaatttgtagcataaaattcgactttattgacgttttttttatgatcatatacaaataaaatagttgcgaatgagaactaaatttttcattgtttttagtcatatcatctggctgttgcacgatgatcgcagcatatttttcttcacaacactcatcctataagcacctttcaggactttcgtctgcactttccatatttgatttcatcaattgatttgaatttttatccagtacaatggtttgaatctatacaataaattagtcacagtataaaattatgtctcatcttgaacacacacctgcttaacaaagaaatgaagcactctatcaatagtaaatacaatatatgcatcgatgccaaatatatatcgtacattctcttcaaacgtagcttgatctaaatcgccatcaataaacctttcaatcatcctcactagtgcattataatggc

SEQ ID NO: 6 - DNA, 729 bp, Rhizopus delemar
ggttactgaaactggtgtagaaattttaacatagaagcaataaaacttacattcttccctctctttacatctacgcatgtttgccaagaataaaatgcctcatttcgggtatatcttttttagataggagttgtaacttgtaatattaagcaatgtaacggacttgtatatttttaatcaaatacgtgtcgtgttttattaaacgagcagaatcacttttctatactgaataaaagtaaaaagaaccgaatgcgttggtaataaattattttatcttttttttttgttttttcacttgtaaaaaaccttaaatgtttaggtttcatccgttttcggcttagtaaaataagctaaaagggcttgtggcgttacacggctattaaactttacataaagtaaaacatcatttgtccctttataacaaaacgaatgggaaaataaccctccatctttttaaacaaaatgagaaaggtaacaataacaaaaactggaatgtgcttgggaaacttgaaaaaaagataagcgcgtcactcttgttttcttttttgtttggttatgcataatattatttgtacctcgaaaaaaaatcttcggaatttgtattttttatatatacggatgatagatgaaaaaaggaaaaagctgatggggcgtatgtatttgtcaaaaaagctataaaagagcacaagttttcacatttcgttttttttttttcttcatactctaacc

SEQ ID NO: 7 - DNA, 955 bp, Rhizopus delemar
ataacataatctcgtaaataacctttttcgctccctgtgaaattatataaataaaaaaaaatcttgtttgtacaaactacttttatgtttcatctttttttttttaaaaaaaaaacgcgttttgattataagcagagatggatttcatccaaaaggaaaattagaaaaatagtctggtcattttgaaaggaaaagaatgcttccaagtaagggttgtttcaacgaagggtataaatcctttcacacgctttcatactttatacatcaaatagtaaggagacttgataaatgccctgtctcaaatgcttttttactttcatgtaaaataagcttgtcttacgcgcaattttactaaccttaagaaggcgattctttgacgatgtttcccgggtattgaacaatcgtttttatttttcgagaacccctgaatctccaaaaaaaggaaggtagttttgaaaaagtatgccgtcatttttactgtaccagtaaaaaaaaatattcttaaacggcatctttgttttacagaaaagcaagaaagcttctcgactccatttttttatagcaccaaatgtcgattacacaagaaagcttaaaataatcatgacaatttccttgaaaataaactttattatagttgaatatagagagctataaaatagctttgcatttccttcttttttttttcttacgattgaaatggtttatttcaaaaggtccatacaccaaataaaaagccatgtataaaagaaaacgtttagggccatcaacaaatataggtattacgcactcaagaaagaacagacggggaaagaccgatatgagtttaacgtccgaaagaaaagaaagggaaaagcccaagactcgaagggcgagaaatagtgcgtttgttaaataaagagtgagaggatactgctaaaaagcttaaagaaatagggcagtgagtagagtgaaaagg

SEQ ID NO: 8 - DNA, 46 bp, Artificial
  primer oJK162
cgagctcgaattatttaaatgaacagcaagttaataatctagaggg

SEQ ID NO: 9 - DNA, 39 bp, Artificial
  primer oJK163
tatgaccatgattacgatgagaggcaaaatgaagcgtac

SEQ ID NO: 10 - DNA, 30 bp, Artificial
  primer oJK164
atttaaataattcgagctcggtacccgggg

SEQ ID NO: 11 - DNA, 20 bp, Artificial
  primer oJK165
cgtaatcatggtcatagctg

SEQ ID NO: 12 - DNA, 29 bp, Artificial
  primer oJK202
tagagggaaaaagagagaattgaaatagg

SEQ ID NO: 13 - DNA, 31 bp, Artificial
  primer oJK204
ttttgttatttaattgtattaattgataatg

SEQ ID NO: 14 - DNA, 36 bp, Artificial
  primer oJK205
aattaaataacaaaatcattttaattacgcattttc

SEQ ID NO: 15 - DNA, 37 bp, Artificial
  primer oJK216
catgattacgcggccgcgccattataatgcactagtg

SEQ ID NO: 16 - DNA, 35 bp, Artificial
  NK-411
cattttgcctctcatggttactgaaactggtgtag

SEQ ID NO: 17 - DNA, 30 bp, Artificial
  NK-412
acgagattatgttatggttagagtatgaag

SEQ ID NO: 18 - DNA, 30 bp, Artificial
  NK-413
ataacataatctcgtaaataacctttttcg

SEQ ID NO: 19 - DNA, 35 bp, Artificial
  NK-414
tatgaccatgattacccttttcactctactcactg

SEQ ID NO: 20 - DNA, 38 bp, Artificial
  primer oJK210
ctctttttccctctaatgagaggcaaaatgaagcgtac

SEQ ID NO: 21 - DNA, 44 bp, Artificial
  primer oJK211
aattaaataacaaaaatgtcttctatcgaaacctccaaaatctc

SEQ ID NO: 22 - DNA, 35 bp, Artificial
  primer NK-052
aattaaataacaaaaatgaccaagaacgttcttac

SEQ ID NO: 23 - DNA, 35 bp, Artificial
  primer NK-064
gcgtaattaaaatgattaagcttttgagctcaaag

SEQ ID NO: 24 - DNA, 25 bp, Artificial
  primer NK-011
ttttgttatttaattgtattaattg

SEQ ID NO: 25 - DNA, 21 bp, Artificial
  primer NK-012
tcattttaattacgcattttc

SEQ ID NO: 26 - DNA, 35 bp, Artificial
  primer NK-274
cttcatactctaaccatgaccaagaacgttcttac

SEQ ID NO: 27 - DNA, 35 bp, Artificial
  primer NK-278
acgagattatgttatttaagcttttgagctcaaag

SEQ ID NO: 28 - DNA, 15 bp, Artificial
  primer NK-269
ggttagagtatgaag

SEQ ID NO: 29 - DNA, 23 bp, Artificial
  primer NK-270
ataacataatctcgtaaataacc

SEQ ID NO: 30 - DNA, 21 bp, Artificial
  primer oJK438
gttccttgctgtggatttgtg

SEQ ID NO: 31 - DNA, 21 bp, Artificial
  primer NK-299
gaaatgtgaaaacttgtgctc

(19) United States
(12) Patent Application Publication
(10) Pub. No.: US 2019/0284586 A1
(43) Pub. Date: Sep. 19, 2019

(54) Filamentous Fungus Variant and C4-Dicarboxylic Acid Production Method Using Same

(71) Applicant: Kao Corporation, Chuo-ku, Tokyo (JP)

(72) Inventor: Kyoshiro NONAKA, Wakayama-shi, Wakayama (JP)

(73) Assignee: Kao Corporation, Chuo-ku, Tokyo (JP)

(21) Appl. No.: 16/463,075

(22) PCT Filed: Nov. 27, 2017
(86) PCT No.: PCT/JP2017/042329

§ 371 Date: May 22, 2019

(30) Foreign Application Priority Data
Dec. 1, 2016 (JP) 2016-234380

(51) Int. Cl. C12P 7/46; C12N 9/08

(52) U.S. Cl. CPC C12P 7/46; C12Y 111/01006; C12N 9/0065

(57) ABSTRACT

Provided are a filamentous fungus mutant strain having an effect of improving C4 dicarboxylic acid productivity, and a method for producing C4 dicarboxylic acid using the filamentous fungus mutant strain. The method for producing a C4 dicarboxylic acid comprises culturing a filamentous fungus mutant strain which has enhanced expression of a polypeptide consisting of the amino acid sequence represented by SEQ ID NO: 2 or an amino acid sequence having at least 90% identity thereto and having catalase activity is enhanced.

### FIELD OF THE INVENTION

The present invention relates to a filamentous fungus mutant strain and a method for producing a C4 dicarboxylic acid using the same.

### BACKGROUND OF THE INVENTION

C4 dicarboxylic acids are utilized not only in various applications in the food industry as an acidulant, an antimicrobial agent and a pH adjusting agent, but also used as a raw material for synthetic resins and biodegradable polymers. Thus, C4 dicarboxylic acids are industrially valuable substances. C4 dicarboxylic acids are industrially produced by either chemical synthesis from petrochemical raw materials or microbial fermentation. Previously, C4 dicarboxylic acids have been mainly produced by chemical synthesis due to a lower cost. However, from the viewpoint of rising costs of the raw materials, the burden on the environment, and the like, production methods by microbial fermentation using a recyclable resource as a raw material have recently been attracting attention.

It is known that fumaric acid, which is one of C4 dicarboxylic acids, can be produced by using a fermentation fungus, such as a Rhizopus. Rhizopus produces fumaric acid using glucose as a carbon source, and excretes the produced fumaric acid to the outside of the cell. To date, as techniques for producing fumaric acid with high productivity by using Rhizopus, improvements of culturing methods, and preparations of strains having high productivity by mutation breeding are known. However, since the genetic background of Rhizopus has not yet been well studied, the development of the techniques for producing fumaric acid with high productivity by Rhizopus through gene recombination is not easy and has little information. There are only a few reports for improving fumaric acid productivity by introducing a gene encoding pyruvate carboxylase from Saccharomyces cerevisiae into Rhizopus delemar (Patent Literature 1), or by introducing a gene encoding phosphoenolpyruvate carboxylase from E. coli into Rhizopus oryzae (Non Patent Literature 1).

[Patent Literature 1] CN-A-103013843

[Non Patent Literature 1] Metabolic Engineering, 2012, 14: 512-520

### SUMMARY OF THE INVENTION

The present invention relates to the following [1] to [4].
[1] A method for producing a C4 dicarboxylic acid comprising culturing a filamentous fungus mutant strain which has enhanced expression of a polypeptide consisting of the amino acid sequence represented by SEQ ID NO: 2 or an amino acid sequence having at least 90% identity thereto and having catalase activity.
[2] A filamentous fungus mutant strain which has enhanced expression of catalase.
[3] A method for producing a filamentous fungus mutant strain comprising introducing a polynucleotide selected from the following 1) to 4) or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.
[4] A method for improving C4 dicarboxylic acid productivity, comprising introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.

### DETAILED DESCRIPTION OF THE INVENTION

The present invention relates to a filamentous fungus mutant strain having an effect of improving C4 dicarboxylic acid productivity, and a method for producing a C4 dicarboxylic acid using the filamentous fungus mutant strain.

The present inventor has conducted intensive studies for producing a C4 dicarboxylic acid using filamentous fungi, and found that the filamentous fungal strain in which expression of catalase has been enhanced has improved C4 dicarboxylic acid productivity.

The filamentous fungus mutant strain of the present invention has improved C4 dicarboxylic acid productivity, and thus can produce a C4 dicarboxylic acid more rapidly. Therefore, the filamentous fungus mutant strain of the present invention is useful for biological production of a C4 dicarboxylic acid. These and other features and advantages of the present invention will become more apparent from the following description of the present specification.

### 1. Definition

In the specification, the identity of amino acid sequences or nucleotide sequences is calculated in accordance with the Lipman-Pearson method (Science, 1985, 227: 1435-1441). Specifically, the identity of amino acid sequences or nucleotide sequences is calculated by using homology analysis (Search homology) program of genetic information processing software GENETYCS Ver. 12 and assigning 2 to the Unit size to compare (ktup).

In the specification, “at least 90% identity” regarding amino acid sequences or nucleotide sequences refers to an identity of 90% or more, preferably 95% or more, more preferably 96% or more, further preferably 97% or more, further preferably 98% or more, further preferably 99% or more.

In the specification, “amino acid sequence having deletion, substitution, addition or insertion of one or more amino acids” refers to an amino acid sequence in which 1 or more and 10 or less, preferably 1 or more and 8 or less, more preferably 1 or more and 5 or less, further preferably 1 or more and 3 or less amino acids are deleted, substituted, added or inserted. Also, in the specification, “nucleotide sequence having deletion, substitution, addition or insertion of one or more nucleotides” refers to a nucleotide sequence in which 1 or more and 30 or less, preferably 1 or more and 24 or less, more preferably 1 or more and 15 or less, further preferably 1 or more and 9 or less nucleotides are deleted, substituted, added or inserted. In the specification, the “addition” of an amino acid or a nucleotide includes addition of the amino acid or nucleotide to one and both ends of a sequence.

In the specification, “upstream” and “downstream” regarding a gene refer to upstream and downstream in transcription direction of the gene. For example, “a gene arranged downstream of a promoter” means that the gene is present at the 3′ side of the promoter in a DNA sense strand, and upstream of a gene refers to the region of the 5′ side of the gene in the DNA sense strand.

In the specification, “operably linking” between a gene and a regulatory region refers to the linking of the gene to the regulatory region such that the gene can be expressed under the control of the regulatory region. A procedure for “operably linking” between a gene and a regulatory region is well known to those skilled in the art.

In the specification, the term “originally” used for function, property or trait of a microorganism means that the function, property or trait are present in the wild-type of the microorganism. In contrast, the term “exogenous” is used to represent that the function, property or trait are not originally present in the microorganism, but externally present in the microorganism. For example, an “exogenous” gene or polynucleotide is a gene or polynucleotide introduced into a microorganism from the outside. The exogenous gene or polynucleotide may be derived from a homogenous biological species of the microorganism in which the gene or polynucleotide is introduced or may be derived from different biological species (more specifically, a heterologous gene or polynucleotide).

In the specification, the “C4 dicarboxylic acid productivity” of a microorganism is represented as a production speed of a C4 dicarboxylic acid in a culture medium of the microorganism. More specifically, a value (g/L/h) obtained by dividing the mass of the C4 dicarboxylic acid per medium volume produced by the cells during a certain culture time elapsed after the start of culture, by the culturing time. The amount of a C4 dicarboxylic acid produced by a microorganism can be calculated as the amount of the C4 dicarboxylic acid in the culture supernatant, which is obtained by removing cells from a culture broth of the microorganism. The amount of a C4 dicarboxylic acid in the culture supernatant can be measured by high performance liquid chromatography (HPLC) or the like. The measurement procedure will be more specifically described in Reference Example 1 described below.

In the specification, “improvement of C4 dicarboxylic acid productivity” in a filamentous fungus mutant strain means that C4 carboxylic acid productivity of the mutant strain has been improved as compared to that of a host or control. The improvement rate of C4 dicarboxylic acid productivity in a filamentous fungus mutant strain is calculated in accordance with the following equation.

Improvement rate (%)=(C4 dicarboxylic acid productivity in mutant strain/C4 dicarboxylic acid productivity in host or control)×100−100

The mutant strain herein refers to a microorganism obtained by modifying a host microorganism such that a predetermined trait is changed. The host refers to a microorganism to which a mutation is to be introduced (parent microorganism strain). Examples of a control include a microorganism in which the same modification as the mutant strain has been made but which is a different species from the host, and a host in which the modification has not been made (e.g., a host to which a vector only or a control sequence has been introduced).

Preferably, the improvement rate of C4 dicarboxylic acid productivity in a filamentous fungus mutant strain is calculated based on the C4 dicarboxylic acid productivity of each filamentous fungus at the time when the C4 dicarboxylic acid-producing speed of the filamentous fungus mutant strain reaches a maximum. In the specification, “a mutant strain having improved C4 dicarboxylic acid productivity by X % or more” refers to a mutant strain exhibiting improvement rate of C4 dicarboxylic acid productivity, calculated in accordance with the above equation being X % or more. Further, in the specification, the “improvement of C4 dicarboxylic acid productivity by X % or more” in a filamentous fungus means that improvement rate of C4 dicarboxylic acid productivity of the filamentous fungus calculated in accordance with the above equation is X % or more.

Examples of the C4 dicarboxylic acid to be produced in the present invention include fumaric acid, malic acid and succinic acid, preferably fumaric acid and malic acid, more preferably fumaric acid.

In the specification, “catalase” means an enzyme (EC 1.11.1.6) that catalyzes disproportionation reaction of hydrogen peroxide (H₂O₂) to oxygen (O₂) and water (H₂O). The “catalase activity” refers to the catalytic activity shown by the catalase, and can be measured, for example, by the method shown in Example 2.

### 2. Filamentous Fungus Mutant Strain and a Method for Producing the Same

### (2.1. Filamentous Fungus Mutant Strain)

The filamentous fungus mutant strain of the present invention is a filamentous fungal strain in which expression of catalase is enhanced. In other words, it is a filamentous fungus mutant genetically constructed to enhance expression of catalase in a host filamentous fungus (parent filamentous fungal strain).

In the present invention, the catalase is preferably catalase derived from Rhizopus, more preferably catalase derived from Rhizopus delemar JCM (Japan Collection of Microorganisms/RIKEN) 5557 strain. The catalase is a polypeptide consisting of an amino acid sequence represented by SEQ ID NO: 2.

Accordingly, in a preferred embodiment, the catalase of the present invention is a polypeptide consisting of the amino acid sequence represented by SEQ ID NO: 2 or an amino acid sequence having at least 90% identity thereto and having catalase activity.

Examples of the amino acid sequence having at least 90%, identity to the amino acid sequence represented by SEQ ID NO: 2 include an amino acid sequence having deletion, substitution, addition or insertion of one or more amino acids with respect to the amino acid sequence represented by SEQ ID NO: 2.

Examples of a method for introducing a mutation such as deletion, substitution, addition, or insertion of an amino acid(s) into an amino acid sequence include a method for introducing a mutation such as deletion, substitution, addition, or insertion of a nucleotide(s) into a nucleotide sequence encoding the amino acid sequence. Examples of the method for introducing a mutation into a nucleotide sequence include mutagenesis using chemical mutagens such as ethyl methanesulfonate, N-methyl-N-nitrosoguanidine and nitrous acid, or physical mutagens such as UV, X-ray, gamma ray and ion beam, method for introducing site-directed mutagenesis, and a method described in Dieffenbach et al (Cold Spring Harbor Laboratory Press, New York, 581-621, 1995). Examples of the method for site-directed mutagenesis include a method using Splicing overlap extension (SOE) PCR (Horton et al., Gene 77, 61-68, 1989), ODA method (Hashimoto-Gotoh et al., Gene, 152, 271-276, 1995), and Kunkel method (Kunkel, T. A., Proc. Natl. Acad. Sci. USA, 1985, 82, 488). Alternatively, commercially available site-directed mutagenesis kits such as Site-Directed Mutagenesis System Mutan-SuperExpress Km kit (Takara Bio Inc.), Transformer™ Site-Directed Mutagenesis kit (Clontech Laboratories, Inc.), and KOD-Plus-Mutagenesis kit (TOYOBO CO., LTD.).

### (2.2. Production of Filamentous Fungus Mutant Strain)

The filamentous fungus mutant strain of the present invention is a filamentous fungus mutant genetically constructed to enhance expression of catalase in a host filamentous fungus (parent filamentous fungal strain). Specifically, the filamentous fungus mutant strain of the present invention can be produced by introducing a polynucleotide encoding catalase expressibly or enhancing expression of the polynucleotide in a host filamentous fungus (parent filamentous fungal strain).

As the host filamentous fungus, any fungi in the filamentous form belonging to the subdivisions Eumycota and Oomycota are included (as defined in Hawksworth et al., In, Ainsworth and Bisby's Dictionary of The Fungi, 8th edition, 1995, CAB International, bUniversity, Press, Cambridge, UK). A filamentous fungus is generally characterized by mycelial cell wall composed of chitin, cellulose, glucan, chitosan, mannan, and other complex polysaccharide. Vegetative growth thereof is through extension of mycelium, and carbon metabolism thereof is obligatory aerobic.

Preferred examples of the host filamentous fungus in the present invention include filamentous fungi of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Parasitella, Penicillium, Phanerochaete, Phlebia, Piromyces, Pleurotus, Rhizopus, Schizophyllum, Talaromyces, Thermoascus, Thielavia, Tolypocladium, Trametes, and Trichoderma. Of these, from the viewpoint of C4 dicarboxylic acid productivity, Rhizopus such as Rhizopus delemar, Rhizopus arrhizus, Rhizopus chinensis, Rhizopus nigricans, Rhizopus tonkinensis, Rhizopus tritici, and Rhizopus oryzae are preferred, Rhizopus delemar and Rhizopus oryzae are more preferred, and Rhizopus delemar is further preferred.

In the present invention, examples of the polynucleotide encoding catalase include the following:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.

Examples of the nucleotide sequence having at least 90% identity to the nucleotide sequence represented having deletion, substitution, addition or insertion of one or more nucleotides with respect to the nucleotide sequence represented by SEQ ID NO: 1.

As used herein, the method for introducing a mutation such as deletion, substitution, addition, or insertion of nucleotide into a nucleotide sequence is as described above. The polynucleotide of the present invention may be in the form of single-stranded or double-stranded, or may be DNA or RNA. The DNA may be cDNA or an artificial DNA such as chemical synthesized DNA.

Examples of a technique for introducing the polynucleotide of the present invention expressibly include, for example, introducing a vector or DNA fragment containing the polynucleotide described above into a host filamentous fungus.

As used herein, the vector containing the polynucleotide of the present invention is an expression vector, preferably an expression vector capable of introducing the polynucleotide into a host filamentous fungus and expressing the polynucleotide in the host. Also the vector preferably includes the polynucleotide of the present invention and a regulatory region operably linked to the polynucleotide. The vector may be a vector capable of extrachromosomally and autonomously proliferating replicating, such as a plasmid, or may be a vector to be incorporated intrachromosomally.

Specific examples of the vector include pBluescript II SK(−) (Stratagene), pUC vector such as pUC18/19 and pUC118/119 (Takara Bio Inc.), pET vector (Takara Bio Inc.), pGEX vector (GE healthcare), pCold vector (Takara Bio Inc.), pHY300PLK (Takara Bio Inc.), pUB110 (Mckenzie, T. et al., 1986, Plasmid 15(2): 93-103), pBR322 (Takara Bio Inc.), pRS403 (Stratagene), pMW218/219 (NIPPON GENE CO., LTD.), pRI vector such as pRI909/910 (Takara Bio Inc.), pBI vector (Clontech Laboratories, Inc.), IN3 vector (Inplanta Innovations Inc.), pPTR1/2 (Takara Bio Inc.), pDJB2 (D. J. Ballance et al., Gene, 36, 321-331, 1985), pAB4-1 (van Hartingsveldt W et al., Mol Gen Genet, 206, 71-75, 1987), pLeu4 (M. I. G. Roncero et al., Gene, 84, 335-343, 1989), pPyr225 (C. D. Skory et al., Mol Genet Genomics, 268, 397-406, 2002), and pFG1 (Gruber, F. et al., Curr Genet, 18, 447-451, 1990).

Examples of the DNA fragment containing the polynucleotide of the present invention include a PCR-amplified DNA fragment and a restriction enzyme-cleaved DNA fragment. Preferably, the DNA fragment may be an expression cassette containing the polynucleotide of the present invention and a regulatory region operably linked thereto.

The regulatory region contained in the vector or DNA fragment is a sequence for expressing the polynucleotide of the present invention in a host into which the vector or DNA fragment has been introduced, and examples of the regulatory region include an expression regulatory region such as a promoter and a terminator, and a replicator. The type of the regulatory region can be selected appropriately depending on the type of host cell into which the vector or DNA fragment is introduced. As necessary, the vector or DNA fragment may further contain a selection marker such as an antibiotic resistance gene and an amino acid synthesis-related gene.

Preferably, the regulatory region contained in the vector or DNA fragment is a regulatory region having a higher transcriptional activity (so-called, an enhanced regulatory region) compared to the regulatory region of the polynucleotide encoding catalase in the host genome. Examples of the enhanced regulatory region for Rhizopus include, but are not limited to, ldhA promoter (U.S. Pat. No. B-6,268,189), pgk1 promoter (WO-A-2001/73083), pgk2 promoter (WO-A-2001/72967), pdcA promoter and amyA promoter (Archives of Microbiology, 2006, 186: 41-50), tef and 18S rRNA promoter (US-A-2010/112651), and adh1 promoter (Japanese Patent Application No. 2015-155759). Further examples of the enhanced regulatory region include, but are not limited to, a regulatory region of rRNA operon, and a regulatory region of the gene encoding ribosomal protein.

The polynucleotide and regulatory region of interest contained in the vector or DNA fragment may be introduced into the nucleus or genome of the host. Alternatively, the polynucleotide of interest contained in the vector or DNA fragment may be directly introduced into the host genome, and operably linked to a high expression promoter on the genome. Examples of the technique for introducing the polynucleotide into the genome include a homologous recombination.

To introduce the vector or DNA fragment into the host cell, general transformation methods such as an electroporation method, a transformation method, a transfection method, a conjugation method, a protoplast method, a particle gun method, or an agrobacterium method can be used.

The filamentous fungus mutant strain into which the vector or DNA fragment of interest has been introduced can be selected using a selection marker. For example, when the selection marker is an antibiotic resistance gene, a transformed cell into which the vector or DNA fragment of interest has been introduced can be selected by culturing cells in a medium containing the antibiotic. As another example, when the selection marker is an amino acid synthesis-related gene, the gene is transferred into an amino acid-auxotrophic filamentous fungal strain and thereafter the filamentous fungal strain into which the vector or DNA fragment of interest has been introduced can be selected based on the presence or absence of amino acid auxotrophy as an indicator. Alternatively, introduction of the vector or DNA fragment of interest can also be confirmed by examining the DNA sequence of the mutant strain by PCR or the like.

Examples of the technique for enhancing expression of the polynucleotide of the present invention include a technique for improving transcription amount of the polynucleotide in the host. For example, the technique includes substituting or inserting the enhanced regulatory region for or into a regulatory region of the polynucleotide of interest on the host genome, and operably linking the enhanced regulatory region to the polynucleotide of interest. Examples of the technique for substituting or inserting a genomic region include a method including introducing a DNA fragment containing the enhanced regulatory region and a selection marker polynucleotide sequence into a host, and selecting the strain which has been transformed by homologous or non-homologous recombination or the like.

The filamentous fungal mutant strain of the present invention obtained by the above procedure has improved intracellular catalase activity, as compared to the host filamentous fungus (parent filamentous fungal strain). Preferably, the catalase activity is 1.2 times or more, 1.5 times or more, 2 times or more, further 3 times or more, or even 4 times or more, as large as that of the host.

### (2.3. Improvement of C4 Dicarboxylic Acid Productivity)

The filamentous fungus mutant strain of the present invention has an improved C4 dicarboxylic acid productivity. For example, the C4 dicarboxylic acid productivity of the mutant strain containing the vector or DNA fragment containing the polynucleotide is improved by preferably 10% or more, more preferably 20% or more, further preferably 30% or more compared to the host filamentous fungus (parent filamentous fungus strain).

### 3. Production of C4 Dicarboxylic Acid

The filamentous fungal mutant strain of the present invention has an improved C4 dicarboxylic acid productivity. Therefore, the present invention also provides a method for producing C4 dicarboxylic acid, including culturing the filamentous fungal mutant strain of the present invention. The C4 dicarboxylic acid produced by the production method of the present invention includes fumaric acid, malic acid, and succinic acid, and preferably the C4 dicarboxylic acid is fumaric acid and malic acid, more preferably fumaric acid.

The medium and culture conditions for culturing the filamentous fungal mutant strain can be appropriately selected depending on the type of the host of the filamentous fungal mutant strain. In general, the medium and culture conditions generally used for culturing the host of the filamentous fungal mutant strain can be used.

For example, the culture temperature may be, for example, from 10° C. to 50° C., preferably from 25° C. to 45° C. The culture period is not particularly limited as long as it is the period during which C4 dicarboxylic acid of interest is sufficiently produced, but may be for example from 1 to 240 hours, preferably from 12 to 120 hours, preferably from 24 to 72 hours. It is preferably cultured under stirring or aeration.

The medium for culturing a filamentous fungus may be any medium commonly used. Preferably, the medium is a liquid medium, and the medium may be any one of a synthetic medium, a natural medium, and a semi-synthetic medium obtained by supplementing natural ingredients to a synthetic medium. The medium may be a commercially available medium such as PDB medium (potato dextrose medium; manufactured by Becton, Dickinson and Company, or the like), PDA medium (manufactured by Becton, Dickinson and Company, or the like), LB medium (Luria-Bertani medium; manufactured by NIHON PHARMACEUTICAL CO., LTD (under trade name “Daigo”), or the like), NB medium (Nutrient Broth; manufactured by Becton, Dickinson and Company, or the like), SB medium (Sabouraud medium; manufactured by Oxoid Limited, or the like), and SD medium (Synthetic Dropout Broth; for example, from Clontech Laboratories, Inc.). Generally, the medium contains a carbon source, a nitrogen source, an inorganic salt and the like, however components and composition of the medium can be set appropriately.

Hereinafter, the preferred medium composition for culturing a filamentous fungus will be described in detail. The concentration of each component in the medium described below represents the concentration of onset (at the preparation of medium or at the start of culturing).

Examples of the carbon source in the medium include glucose, maltose, starch hydrolysate, fructose, xylose and sucrose, and, among these, glucose and fructose are preferred. These saccharides may be used alone or in combination of two or more. The concentration of the carbon source in the medium is preferably 1% (w/v) or more, more preferably 5% (w/v) or more, still more preferably 7.5% (w/v) or more, and is preferably 40% (w/v) or less, more preferably 30% (w/v) or less. Alternatively, the concentration of the carbon source in the medium is preferably from 1 to 40% (w/v), more preferably from 5 to 30% (w/v), still more preferably from 7.5 to 30% (w/v).

Examples of the nitrogen source in the medium include nitrogen-containing compounds such as ammonium sulfate, urea, ammonium nitrate, potassium nitrate, and sodium nitrate. The concentration of nitrogen source in the medium may be preferably from 0.001 to 0.5% (w/v), more preferably from 0.001 to 0.2% (w/v).

The medium may contain a sulfate, magnesium salt, and zinc salt. Examples of the sulfate include magnesium sulfate, zinc sulfate, potassium sulfate, sodium sulfate, and ammonium sulfate. Examples of the magnesium salt include magnesium sulfate, magnesium nitrate, and magnesium chloride. Examples of the zinc salt include zinc sulfate, zinc nitrate, and zinc chloride. The concentration of the sulfate in the medium is preferably from 0.001 to 0.5% (w/v), more preferably from 0.001 to 0.2% (w/v). The concentration of the magnesium salt in the medium is preferably from 0.001 to 0.5% (w/v), more preferably from 0.01 to 0.1% (w/v). The concentration of the zinc salt in the medium is preferably from 0.001 to 0.05% (w/v), more preferably from 0.005 to 0.05% (w/v).

The pH of medium (25° C.) is preferably from 3 to 7, more preferably from 3.5 to 6. The pH of the medium can be adjusted with bases such as calcium hydroxide, sodium hydroxide, calcium carbonate, and ammonia, or acids such as sulfuric acid and hydrochloric acid.

Preferred examples of the medium include a liquid medium containing from 7.5 to 30% carbon source, from 0.001 to 0.2% ammonium sulfate, from 0.01 to 0.6% potassium dihydrogen phosphate, from 0.01 to 0.1% magnesium sulfate heptahydrate, from 0.005 to 0.05% zinc sulfate heptahydrate, and from 3.75 to 20% calcium carbonate (wherein any concentration % is represented by % (w/v)).

To produce C4 dicarboxylic acid more efficiently using a filamentous fungus, production may be carried out in the following steps. That is, a C4 dicarboxylic acid may be produced efficiently by preparing a spore suspension of a filamentous fungus (step A), culturing the spore suspension in a culture solution to germinate a spore, thereby preparing a mycelia (step B1), preferably further proliferating the mycelia (step B2), and culturing the prepared mycelia to produce C4 dicarboxylic acid (step C). However, the steps of culturing the mutant filamentous fungus of the present invention are not limited to the following steps.

### <Step A: Preparation of Spore Suspension>

Spores of a mutant filamentous fungus are inoculated, for example, into a medium such as an inorganic agar medium (composition example: 2% glucose, 0.1% ammonium sulfate, 0.06% potassium dihydrogen phosphate, 0.025% magnesium sulfate heptahydrate, 0.009% zinc sulfate heptahydrate, 1.5% agar, wherein concentrations are all expressed by % (w/v)) and PDA medium, and culturing stationary at from 10 to 40° C., preferably from 27 to 30° C. for 7 to 10 days to form spores, which are suspended in saline or the like. The spore suspension may contain mycelia or not.

### <Step B1: Preparation of Mycelia>

The spore suspension obtained in step A is inoculated into the culture solution and cultured to germinate the spore to obtain mycelia. The number of spores of a filamentous fungus to be inoculated into a culture solution is from 1×10² to 1×10⁸ spores/mL-culture solution, preferably from 1×10² to 5×10⁴ spores/mL-culture solution, more preferably from 5×10² to 1×10⁴ spores/mL-culture solution, further preferably from 1×10³ to 1×10⁴ spores/mL-culture solution. As the culture solution, commercially available medium such as PDB medium, LB medium, NB medium, SB medium, and SD medium can be used. From the viewpoint of germinating rate and mycelial growth, the culture solution can be appropriately added with a carbon source including monosaccharides such as glucose and xylose, oligosaccharides such as sucrose, lactose and maltose, and polysaccharides such as starch; biological substances such as glycerin and citric acid; a nitrogen source such as ammonium sulfate, urea or amino acids; and other inorganic substances such as various salts including a sodium salt, a potassium salt, a magnesium salt, a zinc salt, an iron salt, and a phosphate. The preferred concentration of monosaccharides, oligosaccharides, polysaccharides and glycerin is from 0.1 to 30% (w/v), the preferred concentration of citric acid is from 0.01 to 10% (w/v), the preferred concentration of ammonium sulfate, urea and amino acids is from 0.01 to 1% (w/v), and the preferred concentration of the inorganic substance is from 0.0001 to 0.5% (w/v). Into the culture solution, the spore suspension is inoculated, and the obtained solution is cultured for preferably from 24 to 120 hours, more preferably from 48 to 72 hours while being stirred at preferably from 80 to 250 rpm, more preferably from 100 to 170 rpm under control of the culture temperature of from 25 to 42.5° C. The amount of the culture solution subjected to culture may be appropriately adjusted in accordance with the size of the culture container, and may be about from 50 to 100 mL when the container is a 200 mL baffled flask, and about from 100 to 300 mL when the container is a 500 mL baffled flask. This culturing allows inoculated spores to germinate and grow into mycelia.

### <Step B2: Proliferation of Mycelia>

From the viewpoint of improving C4 dicarboxylic acid productivity, it is preferable to perform the step of further culturing the mycelia obtained in step B1 to proliferate (step B2). The culture solution for proliferation used in step B2 is not particularly limited and may be a commonly used-inorganic culture solution containing glucose. Examples of the solution include a culture solution containing from 7.5 to 30% glucose, from 0.001 to 0.2% ammonium sulfate, from 0.01 to 0.6% potassium dihydrogen phosphate, from 0.01 to 0.1% magnesium sulfate heptahydrate, from 0.005 to 0.05% zinc sulfate heptahydrate, and from 3.75 to 20% calcium carbonate (concentrations are all represented by % (w/v)). The amount of culture solution is appropriately adjusted in accordance with the culture container, and for example, may be from 50 to 300 mL, preferably from 100 to 200 mL when the container is a 500 mL Erlenmeyer flask. Into the culture solution, the mycelia cultured in step B1 are inoculated so as to obtain a rate of from 1 to 30 g-mycelia/100 mL-culture solution, preferably from to 25 g-mycelia/100 mL-culture solution as wet weight, and the obtained solution is cultured for preferably from 12 to 120 hours, more preferably from 24 to 72 hours while being stirred at from 100 to 300 rpm, preferably from 170 to 230 rpm under control of the culture temperature of from 25 to 42.5° C.

### <Step C: Production of a C4 Dicarboxylic Acid>

By culturing the mycelia of a filamentous fungus obtained in the above procedure (step B1 or B2), a C4 dicarboxylic acid is produced by the fungus. Conditions of the culture may follow the ordinary culture conditions of a filamentous fungus described above. The amount of medium may be about from 20 to 80 mL when using a 200 mL Erlenmeyer flask, about from 50 to 200 mL when using a 500 mL Erlenmeyer flask, and from 10 to 15 L when using a 30 L jar fermenter. However, the amount of medium may be appropriately adjusted in accordance with the size of the culture container. The inoculation amount of the mycelia obtained in step B1 or B2 to the medium is preferably from 5 g to 90 g-mycelia/100 mL-medium, more preferably from 5 g to 50 g-mycelia/100 mL-medium as wet weight. Suitably, the cell is cultured for from 2 to 240 hours, preferably from 12 to 120 hours, while being stirred at from 100 to 300 rpm, preferably from 150 to 230 rpm under the temperature of 25 to 45° C. When a jar fermenter is used, aeration is carried out preferably at from 0.05 to 2 vvm, more preferably at from 0.1 to 1.5 vvm.

The filamentous fungus mutant strain of the present invention is cultured according to the above procedure to produce a C4 dicarboxylic acid. After culturing, the C4 dicarboxylic acid is collected from the cultured broth. If necessary, the C4 dicarboxylic acid collected may be further purified. Methods for collecting or purifying a C4 dicarboxylic acid from the cultured broth are not particularly limited, and may be performed in accordance with known collection or purification methods. For example, the C4 dicarboxylic acid in the cultured broth can be collected or purified by removing cells or the like from the cultured broth by a method such as a gradient method, filtration, centrifugation, if necessary, concentrating the remaining cultured broth, and then subjecting the concentrate to crystallization, ion exchange, solvent extraction, or combinations of these.

The filamentous fungus mutant strain of the present invention separated from cultured broth can be reused to produce a C4 dicarboxylic acid. For example, it is possible that to the filamentous fungus mutant strain of the present invention separated from the cultured broth, the medium described above is freshly added, then the resultant is cultured again under the above conditions to produce a C4 dicarboxylic acid, and the produced C4 dicarboxylic acid is collected from the medium. This process can be further repeated. In the production method of the present invention, the culturing of the filamentous fungus mutant strain and the collection of a C4 dicarboxylic acid may be conducted in any one of batch mode, semi-batch mode, and continuous mode.

### 4. Exemplary Embodiment

As exemplary embodiments of the present invention, substances, production methods, uses, methods and the like are further disclosed herein as follows. However, the present invention is not limited to these embodiments.
<1> A method for producing a C4 dicarboxylic acid comprising culturing a filamentous fungus mutant strain which has enhanced expression of a polypeptide consisting of the amino acid sequence represented by SEQ ID NO: 2 or an amino acid sequence having at least 90% identity thereto and having catalase activity.
<2> The method according to <1>, further comprising collecting a C4 dicarboxylic acid from the culture.
<3> The method according to <1> or <2>, wherein the C4 dicarboxylic acid is fumaric acid, malic acid or succinic acid.
<4> The method according to any one of <1> to <3>, wherein the filamentous fungus mutant strain is a filamentous fungus mutant strain obtained by introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.
<5> The method according to <4>, wherein the host filamentous fungus is Rhizopus.
<6> A filamentous fungus mutant strain which has enhanced expression of catalase.
<7> The filamentous fungus mutant strain according to <6>, wherein the catalase is catalase derived from Rhizopus.
<8> The filamentous fungus mutant strain according to <6>, wherein the catalase is a polypeptide consisting of the amino acid sequence represented by SEQ ID NO: 2 or an amino acid sequence having at least 90% identity thereto and having catalase activity.
<9> The filamentous fungus mutant strain according to <8>, wherein the strain is obtained by introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.
<10> The filamentous fungus mutant strain according to any one of <6> to <9>, wherein the filamentous fungus is Rhizopus.
<11> The filamentous fungus mutant strain according to <9> or <10>, wherein the introducing the polynucleotide expressibly in the host filamentous fungus is performed by introducing a vector or DNA fragment comprising the polynucleotide into the host filamentous fungus.
<12> The filamentous fungus mutant strain according to <11>, wherein the vector or DNA fragment further comprises a regulatory region operably linked to the polynucleotide.
<13> The filamentous fungus mutant strain according to <11> or <12>, wherein the vector or DNA fragment is introduced into the nuclear or genome.
<14> The filamentous fungus mutant strain according to <11>, wherein the enhancing expression of the polynucleotide in the host filamentous fungus is performed by substituting or inserting an enhanced regulatory region for or into a regulatory region of the polynucleotide in the host genome.
<15> The mutant filamentous fungus according to any one of <6> to <14>, wherein the filamentous fungus is Rhizopus.
<16> The mutant filamentous fungus according to <15>, wherein the Rhizopus is preferably Rhizopus delemar or Rhizopus oryzae, more preferably Rhizopus delemar.
<17> A method for producing a filamentous fungus mutant strain comprising introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.
<18> A method for improving C4 dicarboxylic acid productivity, comprising enhancing expression of catalase in a host filamentous fungus.
<19> The method according to <18>, wherein the catalase is catalase derived from Rhizopus.
<20> The method according to <18> wherein the catalase is a polypeptide consisting of the amino acid sequence represented by SEQ ID NO: 2 or an amino acid sequence having at least 90% identity thereto and having catalase activity.
<21> The method according to any one of <18> to <20>, wherein the enhancing expression of catalase comprises introducing a polynucleotide selected from the following 1) to 4) expressibly or enhancing expression of the polynucleotide in the host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.

### EXAMPLES

Hereinafter, the present invention will be described in more detail based on Examples; however, the present invention is not limited to these Examples.

### Example 1

### Preparation of Mutant Filamentous Fungus

The PCR primers used in this Example are listed in Table 1.

TABLE 1

| | | SEQ ID |
|---|---|---|
| Primer | Sequence (5′→3′) | NO: |
| oJK162 | cgagctcgaattatttaaatgaacagcaagttaataatctagaggg | 8 |
| oJK163 | tatgaccatgattacgatgagaggcaaaatgaagcgtac | 9 |
| oJK164 | atttaaataattcgagctcggtacccgggg | 10 |
| oJK165 | cgtaatcatggtcatagctg | 11 |
| oJK202 | tagagggaaaaagagagaattgaaatagg | 12 |
| oJK204 | ttttgttatttaattgtattaattgataatg | 13 |
| oJK205 | aattaaataacaaaatcattttaattacgcattttc | 14 |
| oJK216 | catgattacgcggccgcgccattataatgcactagtg | 15 |
| NK-411 | cattttgcctctcatggttactgaaactggtgtag | 16 |
| NK-412 | acgagattatgttatggttagagtatgaag | 17 |
| NK-413 | ataacataatctcgtaaataacctttttcg | 18 |
| NK-414 | tatgaccatgattacccttttcactctactcactg | 19 |
| oJK210 | ctctttttccctctaatgagaggcaaaatgaagcgtac | 20 |
| oJK211 | aattaaataacaaaaatgtcttctatcgaaacctccaaaatctc | 21 |
| NK-052 | aattaaataacaaaaatgaccaagaacgttcttac | 22 |
| NK-064 | gagtaattaaaatgattaagcttttgagatcaaag | 23 |
| NK-011 | ttttgttatttaattgtattaattg | 24 |
| NK-012 | tcattttaattacgcattttc | 25 |
| NK-274 | cttcatactctaaccatgaccaagaacgttcttac | 26 |
| NK-278 | acgagattatgttatttaagcttttgagctcaaag | 27 |
| NK-269 | ggttagagtatgaag | 28 |
| NK-270 | ataacataatctcgtaaataacc | 29 |
| oJK438 | gttccttgctgtggatttgtg | 30 |
| NK-299 | gaaatgtgaaaacttgtgctc | 31 |

### (1) Genome Extraction

Into PDA medium, spores of the 5557 strain of Rhizopus delemar JCM (Japan Collection of Microorganisms/RIKEN) 5557 strain (hereinafter, referred to as 5557 strain) were inoculated, and cultured at 30° C. for 5 days. After culturing, the obtained fungus bodies were placed together with metal cones for 3 mL (Yasui Kikai Corporation) in a 3 mL homogenizing tube, and then immediately frozen in liquid nitrogen for 10 minutes or more. After that, the mycelia were homogenized using a multi-beads shocker (Yasui Kikai Corporation) at 1700 rpm for 10 seconds. After crushing, 400 μL of TE Buffer (pH 8.0) (NIPPON GENE CO., LTD.) was added to the container and mixed by inversion, and 250 μL of the obtained mixture was transferred to 1.5 mL tube. From the resultant mycelia solution, a genome was extracted by using “Dr. GenTLE (from Yeast)” (Takara Bio Inc.) according to the Protocol. To 50 μL of the obtained genomic solution, 1 μL of RNaseA (Roche, Ltd.) was added, and reacted at 37° C. for 1 hour. After the reaction, an equal amount of phenol/chloroform was added and mixed by tapping. Thereafter, the obtained mixture was centrifuged at 4° C. for 5 minutes at 14,500 rpm, and the supernatant was transferred to a fresh 1.5 mL tube. The phenol/chloroform treatment was repeated again and then the resultant was subjected to ethanol precipitation to obtain a purified genomic solution of 5557 strain.

(2) Preparation of cDNA

### (i) Extraction of Total RNA

Into 40 mL of liquid medium (0.1 g/L (NH₄)₂SO₄, 0.6 g/L KH₂PO₄, 0.25 g/L MgSO₄.7H₂O, 0.09 g/L ZnSO₄.7H₂O, 50 g/L calcium carbonate, and 100 g/L glucose), 6 g wet weight of mycelia of 5557 strain was inoculated and cultured at 35° C. for 8 hours at 170 rpm. Mycelia were collected from the culture solution by filtration, and washed twice with 100 mL of 0.85% saline. After washing, excess water was removed by suction filtration, and then 0.3 g of the mycelia were weighed out and placed in a 3 mL homogenizing tube together with metal cones for 3 mL (Yasui Kikai Corporation), and then immediately put into liquid nitrogen to freeze. The resulting frozen mycelia were homogenized using a multi-beads shocker (Yasui Kikai Corporation) for 10 seconds at 1,700 rpm. To the homogenized mycelia, 500 μL of RLT buffer was added, mixed by inversion, and then 450 μL of the obtained mixture was subjected to RNeasy Plant Mini Kit (Qiagen) to extract total RNA. To 40 μL of the obtained RNA solution, 1 μL of DNaseI (Takara Bio Inc.) and 5 μL of 10×DNaseI buffer (USB Corporation) were added. The obtained mixture was filled up to 50 μL with RNase free water and reacted at 37° C. for 30 minutes or more to remove residual DNA in the solution. Further 1 μL of DNaseI was added, and the obtained mixture was reacted at 37° C. for 30 minutes, and subjected to phenol/chloroform extraction, followed by ethanol precipitation. The precipitate was dissolved in 50 μL of sterilize water, and the concentration and purity of the RNA solution were determined using Qubit (Life Technologies). Further, the RNA solution was appropriately diluted, and the extracted RNA was assayed using Agilent 2100 Bioanalyzer (Agilent Technologies) and RNA6000 Pico Kit (Agilent Technologies). The resultant RNA solution, which was confirmed to have an RNA degradation index: “RNA Integrity Number (RIN)” of 6.0 or more, was acquired as total RNA.

(ii) Synthesis of cDNA

cDNA was synthesized using SuperScriptIII First-Strand Synthesis SuperMix for qRT-PCR (Invitrogen). Specifically, 1 μg of the RNA solution obtained in (i) was filled up to 8 μL with DEPC water, thereafter 10 μL of 2× RT Reaxtion Mix and 2 μL of RT Enzyme Mix were added to the solution and gently mixed, and then the obtained mixture was reacted at 25° C. for 10 minutes, at 50° C. for 30 minutes, and at 85° C. for 5 minutes. After the reaction, 1 μL of RNaseH was added to the solution, the obtained mixture was reacted at 37° C. for 20 minutes, and the resultant was used as cDNA solution.

### (3) Preparation of Plasmid Vector

(i) Introduction of trpC Gene Region to pUC18

Using the genomic DNA of 5557 strain obtained in (1) above as a template, a DNA fragment containing trpC gene (SEQ ID NO: 3) was synthesized by PCR using primers oJK162 (SEQ ID NO: 8) and oJK163 (SEQ ID NO: 9). Then, using plasmid pUC18 as a template, a DNA fragment was amplified by PCR using primers oJK164 (SEQ ID NO: 10) and oJK165 (SEQ ID NO: 11). The above two fragments were ligated using In-Fusion HD Cloning Kit (Clontech Laboratories, Inc.) to construct a plasmid pUC18-trpC.

### (ii) Cloning of Promoter and Terminator

Using the genomic DNA of 5557 strain obtained in (1) above as a template, a DNA fragment containing a promoter sequence of ADH1 (SEQ ID NO: 4) and a DNA fragment containing a terminator sequence of ADH1 (SEQ ID NO: 5) each were amplified by PCR using a primer pair of oJK202 (SEQ ID NO: 12) and oJK204 (SEQ ID NO: 13) and a primer pair oJK205 (SEQ ID NO: 14) and oJK216 (SEQ ID NO: 15) respectively. Then, a DNA fragment containing a promoter sequence of cipC (SEQ ID NO: 6) and a DNA fragment containing a terminator sequence of cipC (SEQ ID NO: 7) each were amplified by PCR using a primer pair of NK-411 (SEQ ID NO: 16) and NK-412 (SEQ ID NO: 17), and a primer pair of NK-413 (SEQ ID NO: 18) and NK-414 (SEQ ID NO: 19) respectively. Next, using the plasmid pUC18-trpC obtained in (i) as a template, a DNA fragment was amplified by PCR using primers oJK210 (SEQ ID NO: 20) and oJK211 (SEQ ID NO: 21). The above fragments were ligated in the same procedure as in (i) to construct plasmids pUC18-trpC-Padh-Tadh and pUC18-trpC-PcipC-TcipC. In the obtained plasmids, ADH1 promoter and ADH1 terminator or cipC promoter and cipC terminator are arranged downstream of trpC gene region in this order.

(iii) Preparation of Plasmid for Gene Introduction

A DNA fragment containing a gene represented by SEQ ID NO: 1 (hereinafter, referred to as RdCAT1) was amplified from the cDNA solution obtained in (2) by PCR using primers NK-052 (SEQ ID NO: 22) and NK-064 (SEQ ID NO: 23). Next, using the plasmid pUC18-trpC-Padh-Tadh obtained in (ii) as a template, a DNA fragment was amplified by PCR using primers NK-011 (SEQ ID NO: 24) and NK-012 (SEQ ID NO: 25). The above two fragments were ligated in the same procedure as in (i) to construct a plasmid pUC18-trpC-Padh-RdCAT1-Tadh. Using this plasmid as a template, a DNA fragment containing RdCAT1 was amplified by PCR using primers NK-274 (SEQ ID NO: 26) and NK-278 (SEQ ID NO: 27). Then, using the plasmid pUC18-trpC-PcipC-TcipC obtained in (ii) as a template, a DNA fragment was amplified by PCR using primers NK-269 (SEQ ID NO: 28) and NK-270 (SEQ ID NO: 29). The above two fragments were ligated in the same procedure as in (i) to construct a plasmid pUC18-trpC-PcipC-RdCAT1-TcipC. In the obtained plasmid, RdCAT1 gene represented by SEQ ID NO: 1 is inserted between cipC promoter and cipC terminator.

(4) Introduction of Gene into Host Cell

### (i) Preparation of Tryptophan Auxotrophic Strain

A tryptophan auxotrophic strain which was used as a host cell of gene introduction was screened from strains which had been mutated by ion beam irradiation to the 5557 strain. Ion beam irradiation was carried out at the ion irradiation facility of Takasaki Advanced Radiation Research Institute, (TIARA: Takasaki Ion Accelerators for Advanced Radiation Application) of the Japan Atomic Energy Agency. The strain was irradiated with 100 to 1250 Gray of rays using ¹²C⁵⁺ accelerated by AVF cyclotron of 220 MeV. Spores were collected from the irradiated fungal cells, and, among them, a tryptophan auxotrophic strain, Rhizopus delemar 02T6 strain (hereinafter referred to as 02T6 strain) was obtained. The 02T6 strain has deletion of a single base at position 2093 in trpC gene coding region (SEQ ID NO: 3) of full length 2298 bp.

### (ii) Amplification of Plasmid Vector

Using the plasmid vectors pUC18-trpC-Padh-Tadh and pUC18-trpC-PcipC-RdCAT1-TcipC prepared in (3) above, E. coli DH5α strain (NIPPON GENE CO., LTD.) was transformed by competent cell transformation method. The obtained transformed cell was left to stand at 37° C. overnight, and the obtained colony was inoculated into 2 mL of LBamp liquid medium (Bacto Trypton 1%, Yeast Extract 0.5% NaCl 1%, sodium ampicillin 50 μg/mL) and cultured at 37° C. overnight. From the obtained cultured broth, each plasmid vector was purified using High Pure Plasmid Isolation Kit (Roche Diagnostics K.K.).

(iii) Introduction of Plasmid Vector into Host Cell

To 100 μL of gold particle solution (60 mg/mL), 10 μL of the DNA solutions (1 μg/μL) containing the plasmid vectors pUC18-trpC-Padh-Tadh and pUC18-trpC-PcipC-RdCAT1-TcipC obtained in (ii) each were added and mixed, then 40 μL of 0.1 M spermidine was added thereto, and the obtained mixture was stirred well with a vortex. Further 100 μL of 2.5 M CaCl₂ was added thereto, the obtained mixture was stirred well with a vortex for 1 minute and centrifuged for 30 seconds at 6,000 rpm, and the supernatant was removed. To the obtained precipitate, 200 μL of 70% EtOH was added, the obtained mixture was stirred with a vortex for 30 seconds and then centrifuged for 30 seconds at 6,000 rpm, and the supernatant was removed. The obtained precipitate was resuspended in 100 μL of 100% EtOH.

Next, into the spores of 02T6 strain prepared in (i) a gene was introduced by using GDS-80 (Nepa Gene Co., Ltd.) and using the above-described DNA-gold particle solution. The spores having the gene introduced therein were stationary cultured in the inorganic agar medium (20 g/L glucose, 1 g/L ammonium sulfate, 0.6 g/L potassium dihydrogen phosphate, 0.25 g/L magnesium sulphate heptahydrate, 0.09 g/L zinc sulphate heptahydrate, 15 g/L agar) at 30° C. for about a week. A part of the grown fungal cells was scraped off by an inoculating loop, and suspended in TE (pH 8.0) (NIPPON GENE CO., LTD.). The obtained suspension was treated at 95° C. for 15 minutes and nucleic acids were extracted from the transformed strains. PCR reaction was carried out using the obtained nucleic acid as a template and primers oJK438 (SEQ ID NO: 30) and NK-299 (SEQ ID NO: 31), and a strain in which introduction of the DNA fragment of interest was confirmed was selected as a transformed strain. A strain having the introduced pUC18-trpC-PcipC-RdCAT1-TcipC containing DNA in which RdCAT1 gene was ligated downstream of cipC promoter was obtained as CAT1. Meanwhile, a strain having the introduced plasmid vector pUC18-trpC-Padh-Tadh containing DNA in which RdCAT1 gene was not inserted was obtained as negative control strain (hereinafter referred to as NC strain). The remaining fungal cells were scraped off by an inoculating loop, and were vigorously mixed in a spore collection solution (8.5 g/L sodium chloride, 0.5 g/L polyoxyethylene sorbitan monooleate). The mixed spore suspension was filtered through 3GP100 cylindrical funnel type glass filter (SIBATA SCIENTIFIC TECHNOLOGY Ltd.) to obtain a spore liquid. The number of spores in the obtained spore liquid was measured using TC20 Automated Cell Counter (Bio-Rad Laboratories, Inc.).

### Example 2

### Measurement of Intracellular Catalase Activity of CAT1 Strain

### (1) Culture of Strain

### (i) Preparation of Mycelia

A 200 mL baffled Erlenmeyer flask (AGC Inc.) was charged with 100 mL of SD/-Trp medium (Clontech Laboratories, Inc.) added with sorbitan monolaurate (Rheodol SP-L10 (Kao Corporation)) at a final concentration of 0.5% (v/v). Each of the spore solutions of CAT1 strain and NC strain prepared in Example 1 was inoculated into the medium at 1×10³ spores/mL-medium and then cultured under stirring at 170 rpm at 27° C. for three days. The obtained cultured broth was filtered with a pre-sterilized stainless steel sieve having a 250 μm-mesh (AS ONE Corporation) to collect fungal cells on the filter.

### (ii) Proliferation of Mycelia

To 100 mL of an inorganic culture solution (0.1 g/L (NH₄)₂SO₄, 0.6 g/L KH₂PO₄, 0.25 g/L MgSO₄.7H₂O, 0.09 g/L ZnSO₄.7H₂O, 50 g/L calcium carbonate, and 100 g/L glucose) charged in a 500 mL volume Erlenmeyer flask, 5.0 to 8.0 g of wet fungal cells collected in (i) were inoculated and cultured under stirring at 220 rpm at 27° C. for about 40 hours. The obtained cultured broth was filtered with previously sterilized stainless screen filter holder (MILLIPORE) to collect fungal cells on the filter. Further the fungal cells were washed with 200 mL of saline on the filter holder. The saline used for washing was removed by suction filtration.

### (2) Preparation of Homogenate of Fungal Cells

6.0 g of wet fungal cells of each CAT1 strain and NC strain obtained in (1) above were inoculated into 40 mL of an inorganic culture solution (0.0175 g/L (NH₄)₂SO₄, 0.06 g/L KH₂PO₄, 0.375 g/L MgSO₄.7H₂O, 0.135 g/L ZnSO₄.7H₂O, 50 g/L calcium carbonate, 100 g/L glucose) charged in 200 mL volume Erlenmeyer flask, and then cultured under stirring at 170 rpm at 35° C. for 24 hours. The obtained cultured broth was filtered with previously sterilized stainless screen filter holder (MILLIPORE) to collect fungal cells on the filter. Further the fungal cells were washed on the filter holder with 200 mL of saline, and the saline was removed by suction filtration. The fungal cells were divided into 0.3 g aliquots and frozen at −80° C. The frozen fungal cells were homogenized using a multi-beads shocker and metal cones (Yasui Kikai Corporation). To this, 1 ml of 50 mM Tris-HCl buffer (pH 8.0) was added, the resultant was homogenized again and centrifuged at 4° C. for 5 minutes at 15,000 rpm, and then the obtained supernatant was concentrated and washed using AmiconUltra-0.5 (10 kDa) to obtain a homogenate of fungal cell.

### (3) Measurement of Catalase Activity

To a 96-well assay plate (UV-STAR) in which 0.1 μL of the homogenate of fungal cells of CAT1 strain and NC strain obtained in (2) above were added, 180 μL of 20 mM phosphate buffer (pH 7.4) was added, and then 20 μL of 0.75% hydrogen peroxide solution was added to initiate a reaction. Based on the slope of absorbance at 240 nm and 27° C. (extinction coefficient=43.6 M⁻¹⋅·cm⁻¹), an activity value (U/mg wet weight of the fungal cells) was calculated using the amount of hydrogen peroxide reduced per minute (U=μmol/min) as a standard. The analysis results are shown in Table 2. Compared to NC strain which is a control strain, the catalase activity of CAT1 strain was improved about 4 folds.

TABLE 2

| | Catalase activity (U/mg wet | Relative |
|---|---|---|
| Sample | weight of the fungal cells) | comparison |
| NC strain | 223 | 1 |
| CAT1 strain | 941 | 4.2 |

### Example 3

### C4 Dicarboxylic Acid Productivity of CAT1 Strain

### (1) Culturing of Strain

### (i) Preparation of Mycelia

The mycelia were proliferated under the same conditions as in Example 2(1)(i).

### (ii) Proliferation of Mycelia

The mycelia were proliferated under the same conditions as in Example 2(1)(ii).

### (2) Evaluation of C4 Dicarboxylic Acid Productivity of Transformed Strains

6.0 g of wet fungal cells of each CAT1 strain and NC strain obtained in (1) above each were inoculated into 40 mL of an inorganic culture solution (0.0175 g/L (NH₄)₂SO₄, 0.06 g/L KH₂PO₄, 0.375 g/L MgSO₄.7H₂O, 0.135 g/L ZnSO₄.7H₂O, 50 g/L calcium carbonate, 100 g/L glucose) charged in a 200 mL volume Erlenmeyer flask, and then cultured under stirring at 170 rpm at 35° C. After culturing for 8 hours, the culture supernatant having no fungal cells was collected, and subjected to quantification of C4 dicarboxylic acid (fumaric acid) by using the procedure described in Reference Example 1 below. Based on the quantified amount of each C4 dicarboxylic acid, the improvement rate of C4 dicarboxylic acid productivity of CAT1 strain was calculated according to the following equation.

Improvement rate (%)=(production speed of CAT1 strain/production speed of NC strain)×100−100

The results are shown in Table 3. Compared to NC strain into which RdCAT1 gene had not been introduced, it was observed that CAT1 strain has improved productivity of fumaric acid by 41%.

TABLE 3

| | C4 dicarboxylic acid | Productivity |
|---|---|---|
| Strain name | production rate (g/L/h) | improvement rate (%) |
| CAT1 strain | 1.40 | 41 |
| NC strain | 0.99 | — |

### Reference Example 1

### Quantification of C4 Dicarboxylic Acid

A C4 dicarboxylic acid in a culture supernatant was quantified by HPLC.

The culture supernatant to be subjected to HPLC analysis was diluted appropriately in advance using 37 mM sulfuric acid. Thereafter, insoluble matter was removed using DISMIC-13cp (0.20 μm cellulose acetate membrane, ADVANTEC) or AcroPrep 96 filter plates (0.2 μm GHP membranes, Pall Corporation).

As an HPLC apparatus, LaChrom Elite (Hitachi High-Technologies Corporation) was used. As an analysis column, a polymer column for organic acid analysis ICSep ICE-ION-300 (7.8 mm I.D.×30 cm, TRANSGENOMC) to which ICSep ICE-ION-300 Guard Column Cartride (4.0 mm I.D.×2.0 cm, TRANSGENOMIC) was connected was used. As an eluent, 10 mM sulfuric acid was used. Elution was conducted at a flow rate of 0.5 mL/min and at a column temperature of 50° C. The C4 dicarboxylic acid was detected by using a UV detector (detection wavelength of 210 nm). A concentration calibration curve was prepared using standard samples [fumaric acid (distribution source code: 063-00655, Wako pure chemical industries)]. Based on the concentration calibration curve, the C4 dicarboxylic acid was quantified.

A value obtained by subtracting the initial amount of C4 dicarboxylic acid in the medium from the amount of quantified C4 dicarboxylic acid in the medium was regarded as the amount of produced C4 dicarboxylic acid. A value obtained by dividing the amount of C4 dicarboxylic acid per culture medium at 8 hours after the start of culturing by the culture time was regarded as the production speed of C4 dicarboxylic acid of the cell.

What is claimed is:

1. A method for producing a C4 dicarboxylic acid comprising culturing a filamentous fungus mutant strain which has enhanced expression of a polypeptide consisting of the amino acid sequence represented by SEQ ID NO: 2 or an amino acid sequence having at least 90% identity thereto and having catalase activity.

2. The method according to claim 1, further comprising collecting a C4 dicarboxylic acid from the culture.

3. The method according to claim 1, wherein the C4 dicarboxylic acid is fumaric acid, malic acid or succinic acid.

4. The method according to claim 1, wherein the filamentous fungus mutant strain is a filamentous fungus mutant strain obtained by introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.

5. The method according to claim 4, wherein the host filamentous fungus is Rhizopus.

6. A filamentous fungus mutant strain which has enhanced expression of catalase.

7. The filamentous fungus mutant strain according to claim 6, wherein the catalase is catalase derived from Rhizopus.

8. The filamentous fungus mutant strain according to claim 6, wherein the catalase is a polypeptide consisting of the amino acid sequence represented by SEQ ID NO: 2 or an amino acid sequence having at least 90% identity thereto and having catalase activity.

9. The filamentous fungus mutant strain according to claim 8, wherein the strain is obtained by introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.

10. The filamentous fungus mutant strain according to claim 6, wherein the filamentous fungus is Rhizopus.

11. A method for producing a filamentous fungus mutant strain, comprising introducing a polynucleotide selected from the following 1) to 4) expressibly or enhancing expression of the polynucleotide in a host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.

12. A method for improving C4 dicarboxylic acid productivity, comprising enhancing expression of catalase in a host filamentous fungus.

13. The method according to claim 12, wherein the catalase is catalase derived from Rhizopus.

14. The method according to claim 12, wherein the catalase is a polypeptide consisting of the amino acid sequence represented by SEQ ID NO: 2 or an amino acid sequence having at least 90% identity thereto and having catalase activity.

15. The method according to claim 12, wherein the enhancing expression of catalase comprises introducing a polynucleotide selected from the following 1) to 4) expressibly or enhancing expression of the polynucleotide in the host filamentous fungus:
1) a polynucleotide encoding a polypeptide consisting of the amino acid sequence represented by SEQ ID NO: 2;
2) a polynucleotide encoding a polypeptide consisting of an amino acid sequence having at least 90% identity to SEQ ID NO: 2 and having catalase activity;
3) a polynucleotide consisting of the nucleotide sequence represented by SEQ ID NO: 1; and
4) a polynucleotide consisting of a nucleotide sequence having at least 90% identity to the nucleotide sequence represented by SEQ ID NO: 1 and encoding a polypeptide having catalase activity.
